(19) United States
(12) Patent Application Publication
(10) Pub. No.: US 2026/0140109 A1
(43) Pub. Date: May 21, 2026

(54) FLEXIBLE MOLECULARLY IMPRINTED SENSOR FOR IN-SITU AND IN-VIVO DETECTION OF GAMMA-AMINOBUTYRIC ACID CONTENT IN PLANTS, AND PREPARATION METHOD AND DETECTION METHOD THEREOF

(71) Applicant: Intelligent Equipment Research Center, Beijing Academy of Agriculture and Forestry Sciences, Beijing (CN)

(72) Inventors: Bin LUO, Beijing (CN); Aixue LI, Beijing (CN); Yueyue WANG, Beijing (CN); Wenxin YU, Beijing (CN); Xiaodong WANG, Beijing (CN); Xiaotong JIN, Beijing (CN); Quan CHEN, Beijing (CN); Han ZHANG, Beijing (CN); Kai KANG, Beijing (CN)

(21) Appl. No.: 19/379,365

(22) Filed: Nov. 4, 2025

(30) Foreign Application Priority Data
Nov. 21, 2024 (CN) 202411671039.6

(51) Int. Cl. G01N 33/543; G01N 33/00; G01N 33/94

(52) U.S. Cl. CPC G01N 33/5438; G01N 33/0098; G01N 33/9426; G01N 2600/00

(57) ABSTRACT

Provided are a flexible molecularly imprinted sensor for in-situ and in-vivo detection of gamma-aminobutyric acid content in plants, and a preparation method and a detection method thereof. The flexible molecularly imprinted sensor includes: a flexible substrate and a graphene electrode unit, the graphene electrode unit being arranged on the flexible substrate, where the graphene electrode unit includes a conductive track, a working electrode, a counter electrode, and a reference electrode, where the counter electrode and the reference electrode are respectively located on two sides of the working electrode, and the working electrode is successively provided from inside to outside with a first modification layer, a second modification layer, and a third modification layer, the first modification layer including an AuNPs material, the second modification layer including an Fc-Ni₃(HITP)₂-ZnFe-LDH material, and the third modification layer including a molecularly imprinted polymer (MIP) material.

### CROSS REFERENCE TO RELATED APPLICATION

This patent application claims the benefit and priority of Chinese Patent Application No. 202411671039.6 filed with the China National Intellectual Property Administration on Nov. 21, 2024, and entitled with “FLEXIBLE MOLECULARLY IMPRINTED SENSOR FOR IN-SITU AND IN-VIVO DETECTION OF GAMMA-AMINOBUTYRIC ACID CONTENT IN PLANTS, AND PREPARATION METHOD AND DETECTION METHOD THEREOF”, the disclosure of which is incorporated by reference herein in its entirety as part of the present application.

### TECHNICAL FIELD

The present disclosure relates to the technical field of gamma-aminobutyric acid detection, and particularly relates to a flexible molecularly imprinted sensor for in-situ and in-vivo detection of gamma-aminobutyric acid content in plants, and a preparation method and a detection method thereof.

### BACKGROUND

Gamma-aminobutyric acid (abbreviation GABA) is a non-protein amino acid that is widely present in various plants, including soybean, potato, and lettuce. GABA promotes photosynthesis and both vegetative and reproductive growth of plants, thereby enhancing their resistance to various adversities. In addition, GABA also plays an important role in nitrogen metabolism in plants, participating in the regulation of intercellular signal transmission, helping plants to utilize nitrogen resources more efficiently, and promoting the growth and development of plants. Therefore, detecting GABA content in plants is of great significance for crop breeding and nutritional value evaluation.

At present, the main detection methods for GABA include colorimetry, high-performance liquid chromatography, and gas chromatography. However, these methods have high requirements for the technological level of equipment and operators, making it difficult to achieve rapid and convenient detection. Moreover, these methods are all in-vitro detection, and a complex sampling pre-treatment process may cause significant damage to plants, which can result in a loss of important biological information, making it difficult to meet the requirements of real-time analysis. Meanwhile, traditional rigid electrodes (such as glassy carbon electrodes, platinum electrodes, and gold electrodes) are difficult to adapt to the soft and curved surface of a plant in monitoring, which affects the accuracy and stability of detection. In contrast, flexible electrodes have flexible substrate materials that can better fit onto a surface of a plant leaf, making measurements more accurate while reducing damage to a plant tissue. Therefore, how to design a flexible sensor capable of in-situ and in-vivo detection of GABA in plants has become a technical problem urgently needing to be solved in the field.

### SUMMARY

The present disclosure provides a flexible molecularly imprinted sensor for in-situ and in-vivo detection of gamma-aminobutyric acid content in plants, and a preparation method and a detection method thereof, to solve the problem in the existing technology that gamma-aminobutyric acid (GABA) is difficult to detect rapidly and conveniently.

In a first aspect, the present disclosure provides a flexible molecularly imprinted sensor for in-situ and in-vivo detection of gamma-aminobutyric acid content in plants, including: a flexible substrate and a graphene electrode unit, the graphene electrode unit being arranged on the flexible substrate; where
the graphene electrode unit includes a conductive track, a working electrode, a counter electrode, and a reference electrode, where the counter electrode and the reference electrode are respectively located on two sides of the working electrode, and the working electrode is successively provided from inside to outside with a first modification layer, a second modification layer, and a third modification layer, the first modification layer including an AuNPs material, the second modification layer including an Fc-Ni₃(HITP)₂-ZnFe-LDH material, and the third modification layer including an MIP material.

According to the flexible molecularly imprinted sensor for in-situ and in-vivo detection of gamma-aminobutyric acid content in plants provided in an embodiment of the present disclosure, the reference electrode is provided with an Ag/AgCl ink layer to form an Ag/AgCl reference electrode.

According to the flexible molecularly imprinted sensor for in-situ and in-vivo detection of gamma-aminobutyric acid content in plants provided in an embodiment of the present disclosure, a copolyester insulating layer is provided in a region where the conductive track is located.

According to the flexible molecularly imprinted sensor for in-situ and in-vivo detection of gamma-aminobutyric acid content in plants provided in an embodiment of the present disclosure, the flexible substrate is a copolyester flexible substrate.

In a second aspect, the present disclosure provides a method for preparing the flexible molecularly imprinted sensor for in-situ and in-vivo detection of gamma-aminobutyric acid content in plants, including:
subjecting a PI tape to patterning using a computer-controlled laser direct writing instrument, and subjecting a resulting PI tape to an induction to obtain a graphene electrode unit body on the PI tape;
transferring the graphene electrode unit body on the PI tape to the flexible substrate;
coating an exposed region of the reference electrode with a predetermined amount of Ag/AgCl ink, heating and curing to obtain an Ag/AgCl reference electrode;
preparing a copolyester insulating layer in a region where the conductive track is located; and
modifying the working electrode to obtain an MIP/Fc-Ni₃(HITP)₂-ZnFe-LDH/AuNPs/LIG/Ecoflex electrode.

According to the method for preparing the flexible molecularly imprinted sensor for in-situ and in-vivo detection of gamma-aminobutyric acid content in plants provided in an embodiment of the present disclosure, transferring the graphene electrode unit body on the PI tape to the flexible substrate includes:
coating the graphene electrode unit body on the PI tape with an Ecoflex mixed solution at a predetermined ratio using a spin coater, and subjecting a resulting system to vacuum drying and heat treatment; and
peeling the graphene electrode unit body from the PI tape to obtain the graphene electrode unit on the flexible substrate.

According to the method for preparing the flexible molecularly imprinted sensor for in-situ and in-vivo detection of gamma-aminobutyric acid content in plants provided in an embodiment of the present disclosure, modifying the working electrode to obtain the MIP/Fc-Ni₃(HITP)₂-ZnFe-LDH/AuNPs/LIG/Ecoflex electrode includes:
immersing the electrode in a 0.5 mg/mL to 2.5 mg/mL (preferably 1 mg/mL) of a HAuCl₄ solution to obtain AuNPs by chronoamperometry;
dissolving Fc, Ni₃(HITP)₂, and ZnFe-LDH in a chitosan solution, and coating the working electrode with a resulting 1 μL to 5 μL (preferably 4 μL) of an Fc-Ni₃(HITP)₂-ZnFe-LDH solution to obtain Fc-Ni₃(HITP)₂-ZnFe-LDH/AuNPs/LIG/Ecoflex;
using beta-cyclodextrin as a functional monomer and gamma-aminobutyric acid as a template molecule to prepare an MIP solution; and
immersing the electrode in the MIP solution, and subjecting a resulting immersed electrode to electropolymerization by cyclic voltammetry to form an MIP, then placing a resulting electrode in an NaOH solution to elute the template molecule, to finally obtain an eluted MIP/Fc-Ni₃(HITP)₂-ZnFe-LDH/AuNPs/LIG/Ecoflex electrode.

According to the method for preparing the flexible molecularly imprinted sensor for in-situ and in-vivo detection of gamma-aminobutyric acid content in plants provided in an embodiment of the present disclosure, conditions for the electropolymerization by cyclic voltammetry include: a voltage of −0.4 V to 1.0 V, an electropolymerization cycle number of 30 cycles to 60 cycles (preferably 50 cycles), and a scan speed of 25 mV/s to 120 mV/s (preferably 100 mV/s).

In a third aspect, the present disclosure provides a detection method based on the flexible molecularly imprinted sensor for in-situ and in-vivo detection of gamma-aminobutyric acid content in plants, including:
punching a hole at a setting position on a surface of a plant leaf to be tested;
attaching the flexible molecularly imprinted sensor to a lower surface of the plant leaf to be tested, ensuring that the working electrode being facing a resulting punched position; and
dropping a buffer solution at the punched position, then establishing connection to an electrochemical workstation, and detecting a concentration of gamma-aminobutyric acid by differential pulse voltammetry.

Compared with the existing technology, the flexible molecularly imprinted sensor for in-situ and in-vivo detection of gamma-aminobutyric acid content in plants, and a preparation method and a detection method thereof, provided in some embodiments of the present disclosure, have the following beneficial effects:

Compared with traditional rigid electrochemical sensors, by integrating the graphene electrode unit on the flexible substrate, the flexible molecularly imprinted sensor exhibits superior fitness. The flexible molecularly imprinted sensor can closely fit onto a surface of a plant leaf and still maintain stable electrochemical performance under deformation. By successively arranging, from inside to outside at a detection end of the working electrode, the first modification layer (AuNPs material), the second modification layer (Fc-Ni₃(HITP)₂-ZnFe-LDH material), and the third modification layer (MIP material), the sensor exhibits high sensitivity and high specificity, enabling in-situ and in-vivo detection of gamma-aminobutyric acid in plants. Thus, the flexible molecularly imprinted sensor not only has high sensitivity, but also a wider detection range, a simple fabrication process, and strong adaptability, allowing operation under various environmental conditions.

### BRIEF DESCRIPTION OF THE DRAWINGS

To illustrate the technical solutions in the present disclosure or in the existing technology more clearly, the following will provide a brief introduction to the drawings required for describing the examples or the existing technology. Apparently, the drawings in the following description merely show some examples of the present disclosure, and those of ordinary skill in the art can still derive other drawings from these drawings without creative efforts.

FIG. 1 shows a schematic diagram of the flexible molecularly imprinted sensor for in-situ and in-vivo detection of gamma-aminobutyric acid content in plants provided in an example of the present disclosure.

FIG. 2 shows a schematic flowchart of the method for preparing the flexible molecularly imprinted sensor for in-situ and in-vivo detection of gamma-aminobutyric acid content in plants provided in an example of the present disclosure.

FIG. 3 is a graph showing the preparation process of the LIG/Ecoflex portion of the flexible molecularly imprinted sensor provided in an example of the present disclosure.

FIG. 4 is a schematic diagram showing the modification of the working electrode in the method for preparing the flexible molecularly imprinted sensor provided in an example of the present disclosure.

FIG. 5 is a schematic diagram showing the detection principle of the flexible molecularly imprinted sensor provided in an example of the present disclosure.

FIG. 6 shows a comparative diagram of the flexible molecularly imprinted sensor provided in a comparative example of the present disclosure.

FIG. 7 shows a schematic flowchart of the detection method of the flexible molecularly imprinted sensor for in-situ and in-vivo detection of gamma-aminobutyric acid content in plants provided in an example of the present disclosure.

### REFERENCE SIGNS

10, flexible substrate; 20, graphene electrode unit; 210, working electrode; 220, counter electrode; 230, reference electrode; and 30, conductive track.

### DETAILED DESCRIPTION OF THE EMBODIMENTS

To make the objects, technical solutions, and advantages of the present disclosure clearer, the technical solutions in the present disclosure are described clearly and completely below with reference to the drawings in the present disclosure. Apparently, the examples described are some rather than all of the examples of the present disclosure. On the basis of the examples of the present disclosure, all other examples that can be obtained by those of ordinary skill in the art without creative efforts shall fall within the scope of the present disclosure.

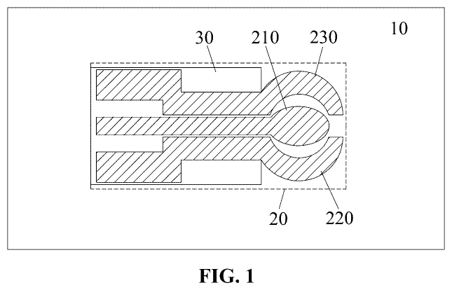

In order to solve the above problems, as shown in FIG. 1, an example of the present disclosure provides a flexible molecularly imprinted sensor for in-situ and in-vivo detection of gamma-aminobutyric acid content in plants, including: a flexible substrate 10 and a graphene electrode unit 20, the graphene electrode unit 20 being arranged on the flexible substrate 10. The graphene electrode unit 20 includes a working electrode 210, a counter electrode 220, a reference electrode 230, and a conductive track 30, the counter electrode 220 and the reference electrode 230 are respectively located on two sides of the working electrode 210, and the working electrode is successively provided from inside to outside with a first modification layer, a second modification layer, and a third modification layer. The first modification layer includes an AuNPs material, the second modification layer includes an Fc-Ni₃(HITP)₂-ZnFe-LDH material, and the third modification layer includes an MIP material.

Compared with traditional rigid electrochemical sensors, by integrating the graphene electrode unit 20 on the flexible substrate 10, the flexible molecularly imprinted sensor for in-situ and in-vivo detection of gamma-aminobutyric acid content in plants in the example of the present disclosure exhibits superior fitness. The flexible molecularly imprinted sensor can closely fit onto a surface of a plant leaf and still maintain stable electrochemical performance under deformation. By successively arranging, from inside to outside on the working electrode 210, the first modification layer (AuNPs material), the second modification layer (Fc-Ni₃(HITP)₂-ZnFe-LDH material), and the third modification layer (MIP material), the sensor exhibits high sensitivity and high specificity. Thus, the flexible molecularly imprinted sensor not only has a wider detection range and a lower detection limit, but also strong adaptability, allowing operation under various environmental conditions, including field applications. In addition, by being combined with smart agriculture application scenarios, the flexible molecularly imprinted sensor can also support unmanned and intelligent real-time dynamic monitoring, providing an efficient and convenient solution for modern agriculture.

Specifically, the flexible substrate 10 is a copolyester flexible substrate (Ecoflex). The copolyester flexible substrate 10 has excellent flexibility and deformability, can remain stable under bending or stretching conditions, and is suitable for close fitting onto a plant surface. In addition, the copolyester flexible substrate 10 exhibits high mechanical strength and durability, capable of withstanding external forces without damage. Further, the copolyester flexible substrate 10 has good chemical stability, and can resist environmental effects such as acid and alkali corrosion and oxidation.

In some embodiments, the graphene electrode unit 20 includes the working electrode 210, the counter electrode 220, the reference electrode 230, and the conductive track 30. The working electrode 210 is the main electrode for electrochemical reactions. The third modification layer of the working electrode is a molecularly imprinted film, which achieves the recognition and detection of GABA based on the principle of molecular imprinting.

In a specific implementation process, the working electrode 210, the counter electrode 220, and the reference electrode 230 are generally connected to an external electrochemical workstation or potential control system via wires. During operation, precise control of voltage and current is required. The external equipment provides current through the counter electrode 220, measures a potential difference through the reference electrode 230, and then undergoes electrochemical reactions with substances in the solution through the working electrode 210.

In some embodiments of the present disclosure, the reference electrode 230 is provided with an Ag/AgCl ink layer to form an Ag/AgCl reference electrode 230.

By providing the Ag/AgCl ink layer on the reference electrode 230 to form the Ag/AgCl reference electrode 230, a stable potential is achieved, maintaining consistency over long periods of time and ensuring reliable and highly reproducible electrochemical results.

As shown in FIG. 1, in some embodiments of the present disclosure, a copolyester insulating layer is provided in a region where the conductive track 30 is located.

By providing the copolyester insulating layer in the region where the conductive track 30 is located, insulation can be achieved in the conductive track region of the graphene electrode unit 20.

A manufacturing and modification process of the working electrode 210 is illustrated as follows.

First, gold nanoparticles (AuNPs) are deposited in a chloroauric acid solution (HAuCl₄) by chronoamperometry (i-t). Subsequently, a mixture of ferrocene (Fc), metal-organic framework Ni₃(HITP)₂, and zinc-iron layered double hydroxide (ZnFe-LDH) is further modified. These nanomaterials can form a network structure with a large specific surface area and have excellent conductivity, amplifying an electrical signal. Then, gamma-aminobutyric acid (GABA) is used as a template molecule, beta-cyclodextrin (beta-CD) is used as a functional monomer, and phosphate buffer solution (PBS) is used to prepare an MIP. Subsequently, a molecularly imprinted polymer (MIP) film is polymerized on a surface of the electrode by cyclic voltammetry (CV). The electrode is then placed into a NaOH solution to elute the GABA template molecule to obtain MIP/Fc-Ni₃(HITP)₂-ZnFe-LDH/AuNPs/LIG/Ecoflex.

Cavities consistent with a shape, structure, and size of GABA are formed on a surface of the MIP film. The cavities can be used to specifically recognize and bind with GABA. After binding with GABA, an electroactive molecule Fc exhibits a decrease in signal, and the decreased current signal correlates with a concentration of GABA. A standard curve for GABA detection is then prepared. Finally, a hole is punched on a leaf surface, the prepared flexible molecularly imprinted sensor is attached to a lower surface of the plant leaf, and an appropriate amount of PBS is dropped so that a sap in the leaf can be released onto the working electrode. A real-time response signal of GABA is recorded by DPV. The flexible molecularly imprinted sensor can specifically bind with GABA. After binding, an oxidation peak current of the electrode decreases in a potential region of the electroactive small molecule Fc, thereby achieving a high-sensitivity detection of GABA. In addition, this method is used for the first time in the GABA detection.

A method for preparing the flexible molecularly imprinted sensor provided in the present disclosure is described below. The method for preparing the flexible molecularly imprinted sensor described below may mutually correspond to and be referenced with the above-described flexible molecularly imprinted sensor for detecting GABA content.

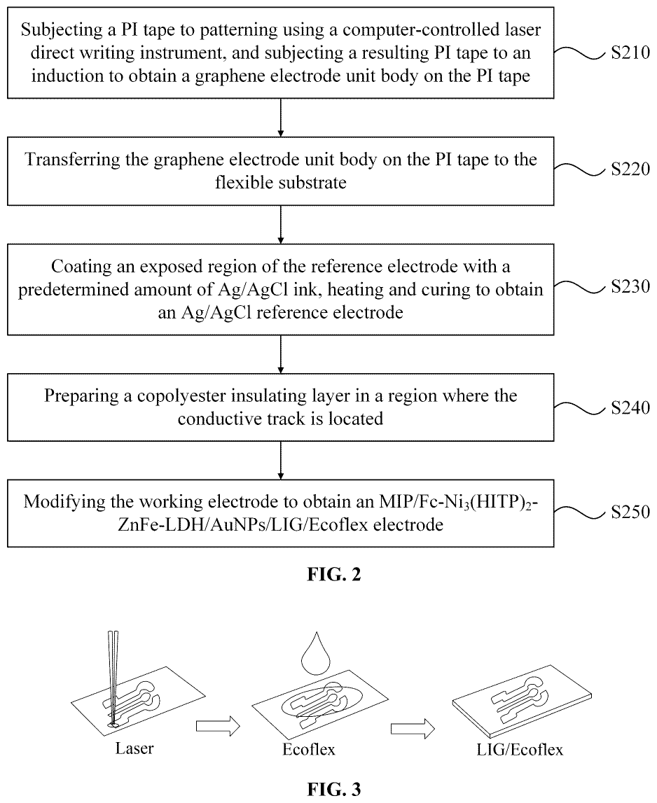

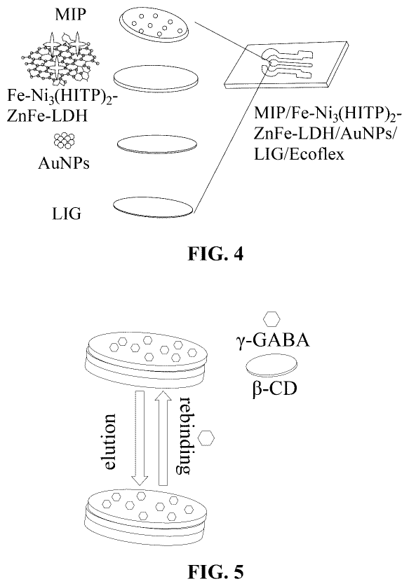

Referring to FIG. 2, FIG. 3, FIG. 4, and FIG. 5, the method for preparing the flexible molecularly imprinted sensor for in-situ and in-vivo detection of gamma-aminobutyric acid content in plants provided in an example of the present disclosure is used for preparing the flexible molecularly imprinted sensor described in the above embodiments, including the following steps:
S210, subjecting a PI tape to patterning using a computer-controlled laser direct writing instrument, and subjecting a resulting PI tape to an induction to obtain a graphene electrode unit body on the PI tape;
S220, transferring the graphene electrode unit body on the PI tape to the flexible substrate;
S230, coating an exposed region of the reference electrode with a predetermined amount of Ag/AgCl ink, heating and curing to obtain an Ag/AgCl reference electrode;
S240, preparing a copolyester insulating layer in a region where the conductive track is located; and
S250, modifying the working electrode to obtain an MIP/Fc-Ni₃(HITP)₂-ZnFe-LDH/AuNPs/LIG/Ecoflex electrode.

Specifically, in step S210, a PI tape is adhered to a polytetrafluoroethylene mold. The PI tape is sequentially cleaned with distilled water and ethanol. Then, a washed PI tape is subjected to patterning using a computer-controlled laser direct writing instrument, and a resulting PI tape is subjected to an induction to obtain a graphene electrode unit body (LIG electrode) with good conductivity.

It should be noted that, at this time, the graphene electrode unit body only includes the working electrode 210, the reference electrode 230, the conductive track 240, the counter electrode 220, and the conductive track 30, and does not include encapsulation layers on the above electrodes and the material layers (the first modification layer, the second modification layer, and the third modification layer) modified on the working electrode 210.

In step S220, an Ecoflex main agent, Ecoflex 00-20, and a curing agent, Ecoflex 00-20, are mixed at a ratio of 15:1 to 20:1 (preferably 10:1) and stirred for 10 min with a spin coater. The mixture is spin-coated at a speed of 50 rpm to 200 rpm (preferably 100 rpm) for 20 s to 80 s (preferably 60 s) to uniformly cover the PI tape with the graphene electrode unit body. Then, a resulting coated PI tape is heated in a vacuum drying oven at a temperature of 60° C. to 100° C. (preferably 80° C.) for 1 h to 5 h (preferably 4 h). The graphene electrode unit body (LIG) is then peeled from the PI tape to obtain the graphene electrode unit 20 (LIG/Ecoflex electrode) on the flexible substrate 10.

That is, the graphene electrode unit 20 is prepared by laser printing on a flexible PI tape according to a design pattern, and then transferring a printed flexible PI tape to a copolyester Ecoflex.

In step S230, an exposed region of the reference electrode 230 is coated with a quantitative amount of Ag/AgCl ink, heated and cured at 80° C. for 10 min to 50 min (preferably 30 min), thereby obtaining the Ag/AgCl reference electrode 230.

In step S240, the region where the conductive track 30 is located is coated with a quantitative amount of Ecoflex, heated and cured to form the copolyester insulating layer, thereby achieving insulation of the region where the conductive track 30 is located. At this point, an LIG/Ecoflex three-electrode system is constructed.

Subsequently, the electrode may be placed in a 0.1 M dilute sulfuric acid solution, and subjected to cyclic voltammetry scanning in a range of 0 V to 1.5 V to perform surface cleaning and activation treatment of the electrode.

Example 1: In step S250, a 0.5 mg/mL to 2.5 mg/mL (preferably 1 mg/mL) of a HAuCl₄ solution was prepared using a phosphate-buffered saline (PBS) solution, and AuNPs were deposited in the HAuCl₄ solution by an i-t method (−1 V, 800 s), obtaining AuNPs/LIG/Ecoflex. Next, a composite material containing 5 g/L to 25 g/L (preferably 10 g/L) of Fc, 0.5 mg/mL to 2 mg/mL (preferably 1 mg/mL) of Ni₃(HITP) 2, and 0.5 mg/mL to 2 mg/mL (preferably 1 mg/mL) of ZnFe-LDH was prepared using a 0.2% chitosan solution. After the composite material was subjected to ultrasonic treatment until uniformly dispersed, 1 μL to 5 μL (preferably 4 μL) of the Fc-Ni₃(HITP)₂-ZnFe-LDH was drop-coated onto a surface of a working electrode, obtaining Fc-Ni₃(HITP)₂-ZnFe-LDH/AuNPs/LIG/Ecoflex. MIP was prepared using PBS, with a molar ratio of GABA:beta-CD of 5:1, where a concentration of GABA was 10 mM to 30 mM (preferably 25 mM) and a concentration of beta-CD was 1 mM to 10 mM (preferably 5 mM). The MIP was formed by electropolymerization through CV (−0.4 V to 1 V, 100 mV/s, 50 cycles), obtaining an uneluted MIP/Fc-Ni₃(HITP)₂-ZnFe-LDH/AuNPs/LIG/Ecoflex. An electrode was then placed into a 10 mM to 60 mM (preferably 50 mM) NaOH solution for 1 min to 10 min (preferably 5 min) to elute the GABA, and finally obtaining an eluted MIP/Fc-Ni₃(HITP)₂-ZnFe-LDH/AuNPs/LIG/Ecoflex.

According to some embodiments of the present disclosure, the method for preparing the flexible molecularly imprinted sensor further included the following steps.

Example 2: A gamma-aminobutyric acid solution with a predetermined concentration was prepared, and a flexible molecularly imprinted sensor was subjected to DPV detection. Based on a relationship curve between the logarithm of a concentration of the gamma-aminobutyric acid solution and a current difference, a standard curve of the flexible molecularly imprinted sensor was plotted.

Specifically, PBS was used to prepare GABA solutions with concentrations of 0, 1 nM, 100 nM, 1 μM, 10 μM, 100 μM, 1 mM, and 10 mM (pH=7.2 to 7.5, preferably pH=7.4), respectively. A resulting prepared flexible molecularly imprinted sensor was used for DPV detection (potential-0.2 V to 0.6 V, interval time 0.5 s, amplitude potential 0.025 V). A peak current of an oxidation peak obtained in a blank solution was recorded as I0, and peak currents obtained for standard gamma-aminobutyric acid solutions of different concentrations were recorded as I1, I2, I3, . . . , respectively. Using the formula ΔI=I0−I1 (I2, I3, . . . ), ΔI1, ΔI2, ΔI3, . . . were respectively calculated. The logarithm of the GABA concentration (lgC_{GABA}) was taken and linearly fitted with ΔI values obtained at different concentrations to plot a standard curve of the GABA flexible molecularly imprinted sensor. A linear equation was ΔI=0.6862 lgC_{GABA}+10.572 (nM), a linear range reached 1×10⁻⁹˜1×10⁻² M, and a detection limit was 5.79×10⁻¹⁰ M.

Example 3: In addition, a recovery rate experiment of the flexible molecularly imprinted sensor was performed, taking lettuce leaves as an example, with specific processes as follows. The lettuce leaves were squeezed into juice, and the juice was centrifuged. After the centrifugation, a supernatant was collected. Based on an established calibration curve, a concentration of GABA in a resulting lettuce juice was about 1.13±0.26 μM. A GABA standard sample was added to this matrix, and a recovery rate was calculated. Experimental results are shown in Table 1, with a spiked recovery rate of GABA being 96.97% to 103.41%. The results were also compared with those obtained by HPLC. A relative error between the results obtained by the prepared sensor and HPLC is 12.4%, which is less than 15%, and therefore considered acceptable. This indicates that the sensor can be used for an accurate determination of GABA in actual samples.

TABLE 1
Spiked recovery rate results (n = 3)

| GABA content | Addition | Test | | Recovery |
|---|---|---|---|---|
| in sample | value | value | RSD | rate |
| (μM) | (μM) | (μM) | (%) | (%) |
| 1.13 ± 0.26 | 1 | 2.09 ± 0.04 | 4.05 | 96.97 |
| (sensor) | | | | |
| 1.29 ± 0.33 | 10 | 10.84 ± 0.29 | 4.71 | 97.34 |
| (HPLC) | | | | |
| | 100 | 104.56 ± 1.13 | 5.82 | 103.41 |

Example 4: Referring to FIG. 7, a detection method of a flexible molecularly imprinted sensor for in-situ and in-vivo detection of gamma-aminobutyric acid content in plants according to an example of the present disclosure included: S710, a hole at a setting position on a surface of a plant leaf to be tested was punched; S720, the flexible molecularly imprinted sensor was attached to a lower surface of the plant leaf to be tested, ensuring that the working electrode was facing a resulting punched position; and S730, a buffer solution was dropwise added at the punched position, then the working electrode was connected to an electrochemical workstation, and a concentration of gamma-aminobutyric acid was detected by differential pulse voltammetry. In a specific implementation process, the plant was any plant containing gamma-aminobutyric acid, including but not limited to fruits, vegetables, flowers, and crops.

An in vivo experiment of the flexible molecularly imprinted sensor was performed, taking lettuce leaves as an example, with specific processes as follows. First, the prepared flexible molecularly imprinted sensor was tested for stability in blank PBS. Then, a hole was punched in a lettuce leaf, and the flexible molecularly imprinted sensor was fixed to a lower surface of the leaf, ensuring that its working electrode was facing a resulting small hole. 20 μL of PBS was dropped into the hole, so that juice contained in the leaf could be released onto the working electrode, and then DPV detection was performed. An obtained current signal was used to calculate an immediate concentration of the tested sample by a corrected working curve.

Two different cultivars of lettuce plants from the same growth period, “Dashusheng” and “Hongshanhu”, were selected. Their GABA contents were detected using the prepared electrode (MIP/Fc-Ni₃(HITP)₂-ZnFe-LDH/AuNPs/LIG/Ecoflex). Detection results are shown in Table 2. The experimental results indicate that the flexible electrode sensor can detect GABA in living plants.

TABLE 2
Comparison list of test results

| | Sample 1 | Sample 2 | Sample 3 | Sample 4 | Sample 5 | Sample 6 | Mean value |
|---|---|---|---|---|---|---|---|
| Cultivar | (μM) | (μM) | (μM) | (μM) | (μM) | (μM) | (μM) |
| Dasusheng | 2.05 | 1.53 | 1.82 | 2.94 | 1.47 | 3.05 | 2.14 ± 0.79 |
| Hongshanhu | 3.14 | 3.53 | 3.82 | 2.84 | 2.77 | 3.75 | 3.31 ± 0.53 |

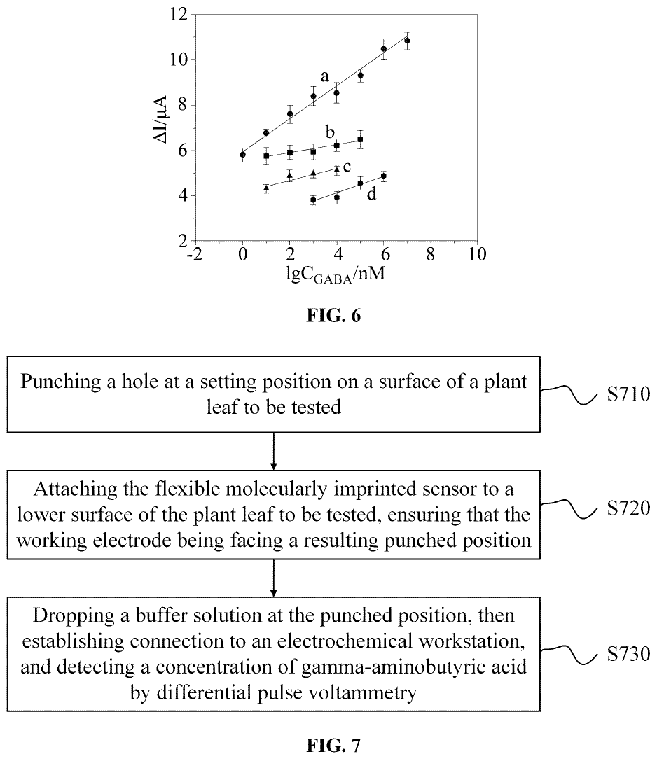

Comparative Example 1: This comparative example provided a flexible molecularly imprinted sensor for detecting GABA, a preparation method of which differed from that of Example 1 (a of FIG. 6) in that: Ni₃(HITP)₂ was replaced with an equivalent amount of Cu(INA)₂ to prepare a flexible molecularly y imprinted sensor b: MIP/Fc-Cu(INA)₂-ZnFe-LDH/AuNPs/LIG/Ecoflex. Detection performance of the sensor was tested using the method in Example 2, and results are shown in b of FIG. 6. It can be seen that the flexible molecularly imprinted sensor prepared in Comparative Example 1 has a linear detection range of 1×10⁻⁸ M to 1×10⁻⁴ M, and a detection limit of 7.74×10⁻⁹ M. The detection effect is not as good as that of Example 1.

Comparative Example 2: This comparative example provided a flexible molecularly imprinted sensor for detecting GABA, a preparation method of which differed from that of Example 1 only in that: ZnFe-LDH was replaced with an equivalent amount of CoFe-LDH to prepare a flexible molecularly imprinted sensor c: MIP/Fc-Ni₃(HITP)₂-CoFe-LDH/AuNPs/LIG/Ecoflex. Detection performance of the sensor was tested using the method in Example 2, and results are shown in c of FIG. 6. It can be seen that the flexible molecularly imprinted sensor prepared in Comparative Example 1 has a linear detection range of 1×10⁻⁸ M to 1×10⁻⁵ M, and a detection limit of 3.21×10⁻⁹ M. The detection effect is not as good as that of Example 1.

Comparative Example 3: This comparative example provided a flexible molecularly imprinted sensor for detecting GABA, a preparation method of which differed from that of Example 1 only in that: AuNPs/LIG/Ecoflex was replaced with LIG/Ecoflex to prepare a flexible molecularly imprinted sensor d: MIP/Fc-Ni₃(HITP)₂-ZnFe-LDH/LIG/Ecoflex. Detection performance of the sensor was tested using the method in Example 2, and results are shown in d of FIG. 6. It can be seen that the flexible molecularly imprinted sensor prepared in Comparative Example 1 has a linear detection range of 1×10⁻⁶ M to 1×10⁻² M, and a detection limit of 6.67×10⁻⁷ M. The detection effect is not as good as that of Example 1.

It should be finally noted that the above examples are only intended to illustrate the technical solutions of the present disclosure but not to limit them. Although the present disclosure has been described in detail with reference to the foregoing examples, those of ordinary skill in the art should understand that they can still make modifications to the technical solutions described in the foregoing examples, or make equivalent substitutions for some of the technical features; and these modifications or substitutions do not cause the essence of the corresponding technical solutions to depart from the spirit and scope of the technical solutions of the examples of the present disclosure.

What is claimed is:

1. A flexible molecularly imprinted sensor for in-situ and in-vivo detection of gamma-aminobutyric acid content in plants, comprising a flexible substrate and a graphene electrode unit, the graphene electrode unit being arranged on the flexible substrate; wherein
the graphene electrode unit comprises a conductive track, a working electrode, a counter electrode, and a reference electrode, wherein the counter electrode and the reference electrode are respectively located on two sides of the working electrode, and the working electrode is successively provided from inside to outside with a first modification layer, a second modification layer, and a third modification layer, the first modification layer comprising an AuNPs material, the second modification layer comprising an Fc-Ni₃(HITP)₂-ZnFe-LDH material, and the third modification layer comprising a molecularly imprinted polymer (MIP) material.

2. The flexible molecularly imprinted sensor for in-situ and in-vivo detection of gamma-aminobutyric acid content in plants of claim 1, wherein the reference electrode is provided with an Ag/AgCl ink layer to form an Ag/AgCl reference electrode.

3. The flexible molecularly imprinted sensor for in-situ and in-vivo detection of gamma-aminobutyric acid content in plants of claim 1, wherein a copolyester insulating layer is provided in a region where the conductive track is located.

4. The flexible molecularly imprinted sensor for in-situ and in-vivo detection of gamma-aminobutyric acid content in plants of claim 1, wherein the flexible substrate is a copolyester flexible substrate.

5. A method for preparing the flexible molecularly imprinted sensor for in-situ and in-vivo detection of gamma-aminobutyric acid content in plants of claim 1, comprising:
subjecting a PI tape to patterning using a computer-controlled laser direct writing instrument, and subjecting a resulting PI tape to an induction to obtain a graphene electrode unit body on the PI tape;
transferring the graphene electrode unit body on the PI tape to the flexible substrate;
coating an exposed region of the reference electrode with a predetermined amount of Ag/AgCl ink, heating and curing to obtain an Ag/AgCl reference electrode;
preparing a copolyester insulating layer in a region where the conductive track is located; and
modifying the working electrode to obtain an MIP/Fc-Ni₃(HITP)₂-ZnFe-LDH/AuNPs/LIG/Ecoflex electrode.

6. The method for preparing the flexible molecularly imprinted sensor for in-situ and in-vivo detection of gamma-aminobutyric acid content in plants of claim 5, wherein transferring the graphene electrode unit body on the PI tape to the flexible substrate comprises:
coating the graphene electrode unit body on the PI tape with an Ecoflex mixed solution at a predetermined ratio using a spin coater, and subjecting a resulting system to vacuum drying and heat treatment; and
peeling the graphene electrode unit body from the PI tape to obtain the graphene electrode unit on the flexible substrate.

7. The method for preparing the flexible molecularly imprinted sensor for in-situ and in-vivo detection of gamma-aminobutyric acid content in plants of claim 5, wherein modifying the working electrode to obtain the MIP/Fc-Ni₃(HITP)₂-ZnFe-LDH/AuNPs/LIG/Ecoflex electrode comprises:
immersing the electrode in a 0.5 mg/mL to 2.5 mg/mL of a HAuCl₄ solution to obtain AuNPs by chronoamperometry;
dissolving Fc, Ni₃(HITP)₂, and ZnFe-LDH in a chitosan solution, and coating the working electrode with a resulting 1 μL to 5 μL of an Fc-Ni₃(HITP)₂-ZnFe-LDH solution to obtain Fc-Ni₃(HITP)₂-ZnFe-LDH/AuNPs/LIG/Ecoflex;
using beta-cyclodextrin as a functional monomer and gamma-aminobutyric acid as a template molecule to prepare an MIP solution; and
immersing the electrode in the MIP solution, and subjecting a resulting immersed electrode to electropolymerization by cyclic voltammetry to form an MIP, then placing a resulting electrode in an NaOH solution to elute the template molecule, to finally obtain an eluted MIP/Fc-Ni₃ (HITP)₂-ZnFe-LDH/AuNPs/LIG/Ecoflex electrode.

8. The method for preparing the flexible molecularly imprinted sensor for in-situ and in-vivo detection of gamma-aminobutyric acid content in plants of claim 7, wherein conditions for the electropolymerization by cyclic voltammetry comprise: a voltage of −0.4 V to 1.0 V, an electropolymerization cycle number of 30 cycles to 60 cycles, and a scan speed of 25 mV/s to 120 mV/s.

9. A detection method based on the flexible molecularly imprinted sensor for in-situ and in-vivo detection of gamma-aminobutyric acid content in plants of claim 1, comprising:
punching a hole at a setting position on a surface of a plant leaf to be tested;
attaching the flexible molecularly imprinted sensor to a lower surface of the plant leaf to be tested, ensuring that the working electrode being facing a resulting punched position; and
dropping a buffer solution at the punched position, then establishing connection to an electrochemical workstation, and detecting a concentration of gamma-aminobutyric acid by differential pulse voltammetry.

10. The method for preparing the flexible molecularly imprinted sensor for in-situ and in-vivo detection of gamma-aminobutyric acid content in plants of claim 5, wherein the reference electrode is provided with an Ag/AgCl ink layer to form an Ag/AgCl reference electrode.

11. The method for preparing the flexible molecularly imprinted sensor for in-situ and in-vivo detection of gamma-aminobutyric acid content in plants of claim 5, wherein a copolyester insulating layer is provided in a region where the conductive track is located.

12. The method for preparing the flexible molecularly imprinted sensor for in-situ and in-vivo detection of gamma-aminobutyric acid content in plants of claim 5, wherein the flexible substrate is a copolyester flexible substrate.

13. The detection method based on the flexible molecularly imprinted sensor for in-situ and in-vivo detection of gamma-aminobutyric acid content in plants of claim 9, wherein the reference electrode is provided with an Ag/AgCl ink layer to form an Ag/AgCl reference electrode.

14. The detection method based on the flexible molecularly imprinted sensor for in-situ and in-vivo detection of gamma-aminobutyric acid content in plants of claim 9, wherein a copolyester insulating layer is provided in a region where the conductive track is located.

15. The detection method based on the flexible molecularly imprinted sensor for in-situ and in-vivo detection of gamma-aminobutyric acid content in plants of claim 9, wherein the flexible substrate is a copolyester flexible substrate.
